(19) United States
(12) Patent Application Publication
(10) Pub. No.: US 2024/0390655 A1
(43) Pub. Date: Nov. 28, 2024

(54) SHAPE SET HYPODERMIC NEEDLE

(71) Applicant: EDWARDS LIFESCIENCES CORPORATION, Irvine, CA (US)

(72) Inventors: Jonathan Andrew Lam, Anaheim Hills, CA (US); Zachary Charles VanEvery, Irvine, CA (US); Tarannum Ishaq Gutierrez, Ladera Ranch, CA (US); Nithya Sridhar, Irvine, CA (US); Lan T. Quach, Irvine, CA (US); Maria G. Verastegui Jaimez, Aliso Viejo, CA (US)

(21) Appl. No.: 18/794,570

(22) Filed: Aug. 5, 2024

Related U.S. Application Data
(63) Continuation of application No. PCT/US2023/012365, filed on Feb. 5, 2023.
(60) Provisional application No. 63/308,454, filed on Feb. 9, 2022.

(51) Int. Cl. A61M 25/09

(52) U.S. Cl. CPC A61M 25/09

(57) ABSTRACT

A device for delivering a guidewire to a target location in a patient's body includes a hollow needle having: a lumen configured to receive the guidewire; a body section; and a biased section, the biased section configured to bend relative to the body section, the biased section located on a distal end of the hollow needle. The device includes a catheter having a catheter lumen, the catheter lumen configured to: receive the hollow needle, and maintain the biased section and the body section in relative alignment in a first configuration. The hollow needle, in response to the biased section extending out from the catheter, is configured to bend the biased section relative to the body section in a second configuration.

### RELATED APPLICATION

This application is a continuation of International Patent Application No. PCT/US2023/012365, filed Feb. 5, 2023, which claims priority to U.S. Provisional Application No. 63/308,454, filed Feb. 9, 2022, and entitled SHAPE SET HYPODERMIC NEEDLE, the disclosures of which are hereby incorporated by reference in their entirety.

### BACKGROUND

### Field

The present disclosure generally relates to the field of implant delivery devices and implant techniques.

### Description of Related Art

Heart failure can occur when the heart becomes enlarged and dilated due to various conditions, such as chronic hypertension, myocardial infarction, mitral valve incompetency, and other dilated cardiomyopathies. Treatments for such heart dysfunctions can involve implanting devices in the heart, either temporarily or permanently. Such devices may need to be delivered to the heart using implant delivery devices that use guidewires, which may be introduced to a chamber of the heart via a hypodermic needle.

### SUMMARY

Described herein are one or more methods and/or devices to facilitate delivery of a guidewire to the heart or other anatomy. The guidewire can then be used to deliver an implant or other medical device. In certain examples, a curved needle can facilitate deliver of the guidewire while avoiding certain issues such as tissue tenting by enabling a smaller profile delivery system for the guidewire.

One general aspect includes a device for delivering a guidewire to a target location in a patient's body. The device includes a hollow needle having: a lumen configured to receive the guidewire; a body section; and a biased section, the biased section configured to bend relative to the body section, the biased section located on a distal end of the hollow needle. The device includes a catheter having a catheter lumen, the catheter lumen configured to: receive the hollow needle, and maintain the biased section and the body section in relative alignment in a first configuration. The hollow needle, in response to the biased section extending out from the catheter, is configured to bend the biased section relative to the body section in a second configuration.

Implementations may include one or more of the following features. The device may include a plurality of cuts made in the biased section that allow the biased section to bend. The plurality of cuts may be uniform and aligned in parallel along a curved inner surface of the biased section. The plurality of cuts may number at least 10 cuts. The plurality of cuts may be laser cuts. The device may include a sealant over the plurality of cuts. The biased section may include a needle tip, where the direction of the needle tip changes by at least 90 degrees when the device transforms from the first configuration to the second configuration. The biased section may have a radius of about 13.5 mm and an arc of about 106 degrees. The biased section may form a u-shape in the second configuration. The device may include a ramp formed within the catheter lumen, the ramp configured to deflect the biased section of the hollow needle relative to the body section. The body section and the biased section may form an angle that is no more than 90 degrees in the second configuration. The catheter may include an opening into the catheter lumen located on its side, the opening configured to provide egress out of the catheter lumen for the hollow needle.

One general aspect includes a method for delivering a guidewire to a target location in a patient's body using a hollow needle deployed from a catheter. The method also includes advancing the hollow needle in the catheter to a first location where the hollow needle includes a biased section and a body section, the biased section and the body section maintained in relative alignment by the catheter in a first configuration. The method also includes advancing the biased section of the hollow needle through an opening of the catheter. The method also includes deploying the biased section of the hollow needle through the opening of the catheter into a second configuration, where in response to the biased section extending out from the catheter, the biased section is configured to bend relative to the body section. The method also includes deploying the guidewire from the hollow needle to the target location.

Implementations may include one or more of the following features. Deploying the biased section of the hollow needle may include piercing through a tissue wall with the hollow needle to reach the target location. The method may include deploying a medical implant to the target location using the guidewire. The biased section may include a needle tip, where the direction of the needle tip changes by at least 90 degrees when transforming from the first configuration to the second configuration. The biased section can form a u-shape in the second configuration. The method may include withdrawing, after deploying the guidewire, the hollow needle from the target location while retaining the hollow needle in the catheter. The hollow needle may be made at least partly from nylon or nitinol.

One general aspect includes a hollow needle for delivering a guidewire to a target location in a patient's body. The hollow needle also includes a lumen configured to receive the guidewire. The needle also includes a body section of the hollow needle. The needle also includes a biased section of the hollow needle located on a distal end of the hollow needle. The biased section may include a needle tip, and a plurality of cuts aligned in parallel along a same side of the biased section where the same side may be an inner surface of a curve formed by the biased section and where the plurality of cuts are configured to cause the biased section to bend relative to the body section.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates human cardiac anatomy.

FIG. 2 illustrates a superior view of a human heart.

FIG. 3 shows an example of tenting, where tissue catches on a needle or part of a needle.

FIGS. 4A, 4B, and 4C show perspective, axial view, and side view, respectively, of a curved needle, in accordance with one or more examples.

FIGS. 4D-4F are cross-sectional views of a needle in a catheter, in a first configuration with the needle within the catheter, a transitionary configuration, and a second configuration with the curved portion of the needle outside of the catheter, respectively, in accordance with one or more examples.

FIG. 5 illustrates another example of a curved needle with a different structure and material, in accordance with one or more examples.

FIGS. 6 and 7 show different examples of a catheter for an implant delivery device, in accordance with one or more examples.

FIG. 8 shows a block diagram of a system for implanting and/or monitoring an implant device, according to one or more examples.

FIGS. 9-1, 9-2, and 9-3 (collectively referred to as FIG. 9), provide a flow diagram representing a process for using an implant delivery device that uses a curved needle to perform a medical procedure, according to one or more examples disclosed herein.

FIGS. 10-1, 10-2, and 10-3 (collectively referred to as FIG. 10) provide a view of the implant delivery device in the heart as the process of FIG. 9 is performed, according to one or more examples disclosed herein.

FIG. 11 illustrates various examples of needle ends (also called needle points) that may be used with examples of the needle described in the above figures, according to one or more examples disclosed herein.

FIGS. 12A-12C illustrate various cut patterns that can be used with examples of the needle described in the above figures, according to one or more examples disclosed herein.

FIG. 13 illustrates a needle that uses a wire stiffener to maintain its shape, according to one or more examples disclosed herein.

FIG. 14 illustrates a needle 1400 that uses a wire stiffener 1406 along its interior curvature to maintain its shape, according to one or more examples disclosed herein.

FIGS. 15A-15C illustrate various examples of wires stiffeners, according to one or more examples disclosed herein.

FIGS. 16A-16C illustrate various views of a device comprising a needle that uses a wire stiffener, and a sheath, according to one or more examples disclosed herein.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard terms of location are used herein to refer to certain device components/features and to the anatomy of animals, and namely humans, with respect to some examples. Although certain spatially relative terms, such as “outer,” “inner,” “upper,” “lower,” “below,” “above,” “vertical,” “horizontal,” “top,” “bottom,” “under,” “over,” “topside,” “underside,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

The present disclosure relates to systems, devices, and methods for delivering implants into a target location in a patient's body, such as the heart. In particular, certain examples utilize a curved, hollow needle for delivering a guidewire to the target location. Using such a needle can reduce the cross-section of at least some parts of an implant delivery device, thereby avoiding or reducing the chance of certain issues, such as tenting. Implant delivery devices of the present disclosure include one or more of a catheter, a tubing within the catheter, a hollow needle, such as a hypodermic needle formed on a distal end of the tubing, and one or more guidewires configured to be delivered to a target site inside the tubing. The guidewires can be used to deliver one or more implants to the target site. In some examples, the hypodermic needle is biased to curve after leaving the catheter. In a first configuration, with the hypodermic needle inside the catheter, the hypodermic needle is largely straight. In a second configuration, with the hypodermic needle outside the catheter, the hypodermic needle is curved. Certain examples of implant delivery devices are disclosed herein in the context of cardiac implant devices. However, although certain principles disclosed herein are particularly applicable to the anatomy of the heart, it should be understood that implant delivery devices in accordance with the present disclosure may be utilized in any suitable or desirable anatomy.

### Cardiac Physiology

The anatomy of the heart is described below to assist in the understanding of certain inventive concepts disclosed herein. In humans and other vertebrate animals, the heart generally comprises a muscular organ having four pumping chambers, wherein the flow between chambers and vessels associated therewith is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

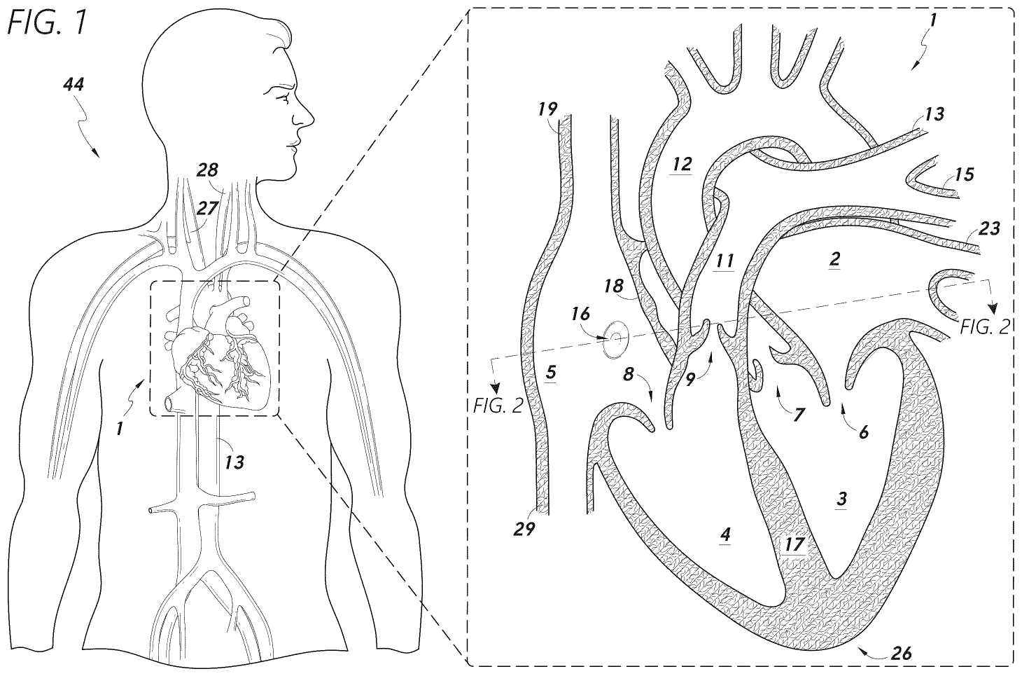

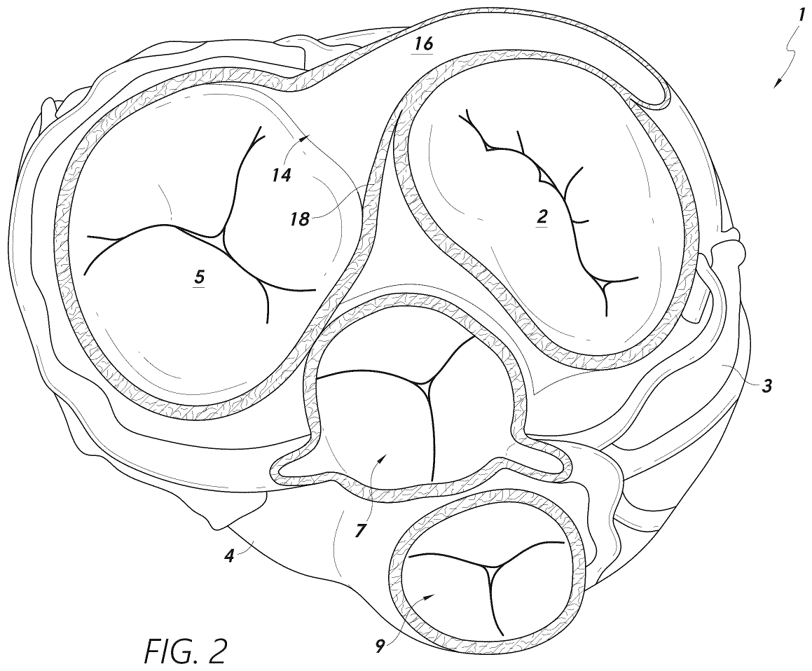

FIGS. 1 and 2 illustrate vertical/frontal and horizontal/superior cross-sectional views, respectively, of an example heart 1 having various features/anatomy relevant to certain aspects of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs.

In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae.

A wall of muscle, referred to as the septum, separates the left-side chambers from the right-side chambers. In particular, an atrial septum wall portion 18 (referred to herein as the “atrial septum,” “atrial septum,” or “septum”) separates the left atrium 2 from the right atrium 5, whereas a ventricular septum wall portion 17 (referred to herein as the “ventricular septum,” “interventricular septum,” or “septum”) separates the left ventricle 3 from the right ventricle 4. The inferior tip 26 of the heart 1 is referred to as the apex and is generally located on or near the midclavicular line, in the fifth intercostal space.

The coronary sinus 16 comprises a collection of veins joined together to form a relatively large vessel that collects blood from the heart muscle (myocardium). The ostium 14 (see FIG. 2) of the coronary sinus 16, which can be guarded at least in part by a Thebesian valve in some patients, is open to the right atrium 5, as shown. The coronary sinus runs along a posterior aspect of the left atrium 2 and delivers less-oxygenated blood to the right atrium 5. The coronary sinus generally runs transversely in the left atrioventricular groove on the posterior side of the heart.

Certain physiological conditions or parameters associated with the cardiac anatomy can impact the health of a patient. For example, congestive heart failure is a condition associated with the relatively slow movement of blood through the heart and/or body, which causes the fluid pressure in one or more chambers of the heart to increase. As a result, the heart does not pump sufficient oxygen to meet the body's needs. The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Acute decompensated congestive heart failure is a leading cause of morbidity and mortality, and therefore treatment and/or prevention of congestive heart failure is a significant concern in medical care. Treatments for such conditions may require implanting various medical devices in the heart.

### Implant Delivery Devices

As referenced above, the treatment of certain cardiac conditions can involve the implantation of devices, such as those designed to shunt blood from one chamber or vessel of the heart to another. Disclosed herein are implant delivery devices that utilize a hollow needle (e.g. a hypodermic needle) that forms a first shape in a first configuration and a second shape in a second configuration. For example, the first shape may be a relatively straight form when the hypodermic needle is inside a catheter. The relatively straight form can facilitate movement of the needle through the catheter. The second shape may be a curved form when the hypodermic needle is released from the catheter. The curved form can facilitate entry into a target location (e.g., a location beside rather than in front of the catheter) and can enable a smaller profile that reduces the chances of catching on a tissue wall. In some examples, the hollow needle comprises a flexible or semi-flexible material that is biased to form a curve when released from the catheter. For example, the hollow needle may be made at least partially of a shape-memory, such as a nickel-titanium alloy (e.g., Nitinol), a copper-aluminum-nickel alloy, iron-based and copper-based alloys, or the like. Other types of material such as nylon or other synthetic polymers can also be used. Such materials can allow the needle to be set in a particular shape or shape set, and return to that shape after being deformed or otherwise changed to a different shape.

For purposes of this disclosure, components of an implant delivery device, as well as sub-components, are described as distal or proximal in terms of direction along the axis of the implant delivery device. Distal components are those that are towards the target tissue site within a patient during an operation, while proximal components are those that are towards the user or machine manipulating the implant delivery device, outside the patient. For example, the needle tip may be at the distal end of the implant delivery device while a catheter handle may be at the proximal end of the implant delivery device.

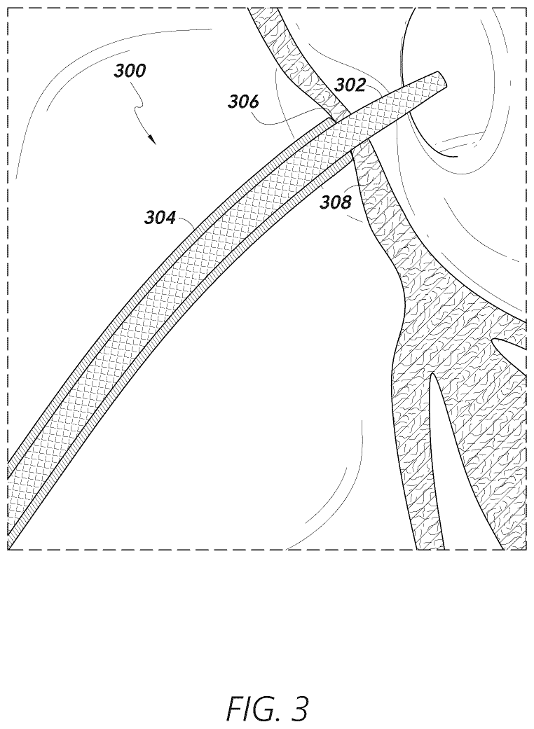

FIG. 3 shows an example of tenting, where tissue catches on a needle or part of a needle. Some implant delivery devices 300 utilize a needle 302 with a jacket 304 covering the needle. The needle is typically solid and is used to pierce into the left atrium 2. After reaching the left atrium, the needle is withdrawn, leaving the jacket 304 within the left atrium. The jacket, with the needle withdrawn, provides a lumen through which a guidewire can then be inserted into the left atrium. The guidewire can then be used to help deliver the implant to the left atrium.

However, using the needle 302 with the jacket 304 enlarges the total diameter of the distal end of the implant delivery device 300. This can create an issue with tenting, as shown in FIG. 3. With the needle extending out of the jacket, the jacket can form a ledge 306 where the distal end of the jacket meets the sides of the needle. This ledge can catch in the tissue of the atrial wall 308 when the needle pierces through. Thus, it can be beneficial to utilize a needle that does not need a jacket.

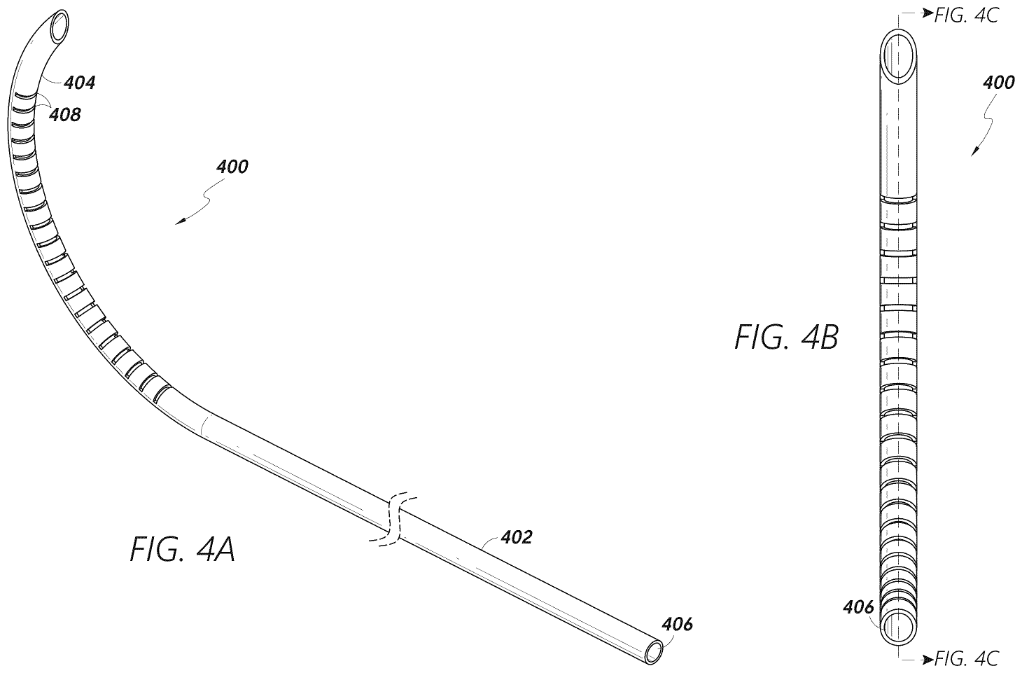

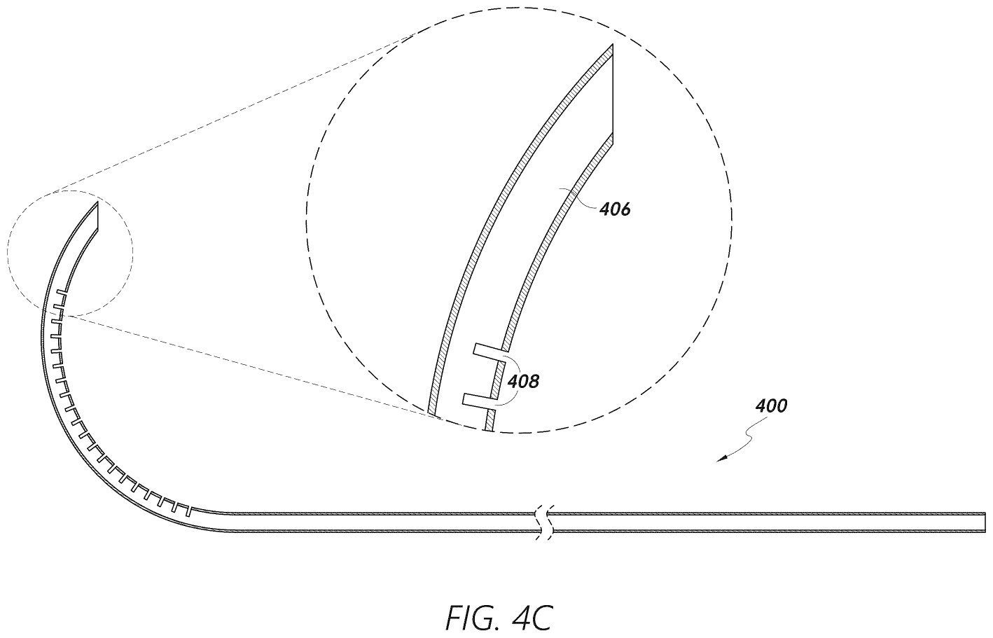

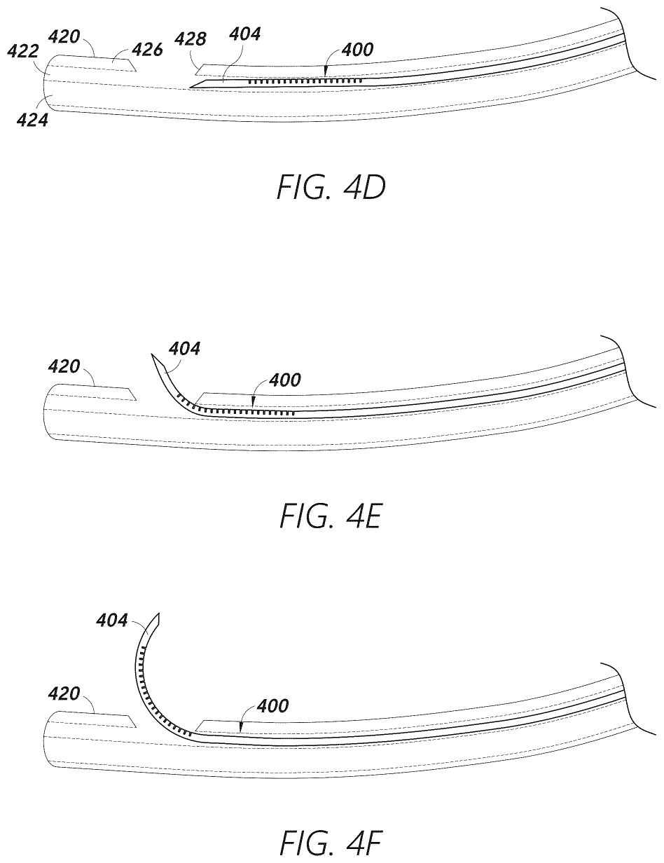

FIGS. 4A-4F show views of a curved needle 400 of an implant delivery device, according to certain examples. In particular, FIG. 4A shows a perspective view while FIG. 4B shows a front facing or axial view. FIG. 4C shows a side view, with an expended view of the needle tip and some of the surface cuts on the needle. FIGS. 4D-4E show the needle in a first configuration in the catheter, then shows the needle transitioning to a second configuration, with the curved portion of the needle outside of the catheter. Unlike the implant delivery device 300 of FIG. 3, the curved needle 400 is used with an implant delivery device that does not need to use a jacket over the needle. Instead, the needle 400 is hollow and the implant delivery device uses the hollow needle 400 to deliver a guidewire to the target location instead of using a tubular jacket to deliver the guidewire.

Focusing on FIGS. 4A-4B, the distal end of the curved needle 400 is shown. The needle 400 can comprise a generally straight needle body 402 and a curved needle end 404 located on distally from the needle body 402. Thus, the needle 400 can include a biased section (needle end 404) that bends relative to a body section (needle body 402), or at least relative to the distal end of the needle body 402 connected to the needle end 404. The needle body 402 section of the needle may extend proximally to a catheter handle and may be much longer than the needle end 404 (e.g., a few centimeters) section of the needle 400. As the needle body 402 can be fairly long (e.g., 10's to 100's of centimeters) and can route through the patient's body, the needle body may not be straight the entire length, but rather have relatively straight portions with curved portions between such straight portions. In some examples, such curved portions of the needle body may change by a few degrees (e.g., no more than 200 or 30°). A guidewire may be inserted into the needle body 402 through the handle or may be pre-inserted into the needle body 402 prior to use on a patient. In the illustrated example, the needle 400 forms a lumen 406 within its body, providing a passageway for sutures to be delivered to a target site. The needle may be produced in different diameters to facilitate different use cases, some of which may require larger lumen sizes. For example, one embodiment of the needle may have an inner diameter of about 1.20 mm and an outer diameter of about 1.52 mm. Another embodiment of the needle may have an inner diameter of about 2.16 mm and an outer diameter of about 2.77 mm. The thickness of the walls of the needle that surround the lumen 406 may also vary depending on the use case and the desired structural strength for the needle.

In an example, the curved needle end 404 includes multiple cuts 408 on one side of its surface. In the illustrated example, the cuts begin a short distance (e.g., 6 mm, 1-10 mm, etc.) from the tip of needle and continue proximally along the curved needle end. The cuts can cause the needle end 404 to curve by creating a bias or tendency for the needle end 404 to bend inwards along the surface where the cuts are made.

FIG. 4C shows a side view of the needle 400. In the illustrated example, the curve is formed by an arc of about 106° with a radius of about 13.5 mm. As will be apparent, various examples of the needle can have different arcs and radii as needed. For example, the curved needle end 404 may be longer (e.g., radius greater than 13.5 mm and/or arc greater than 106°) or shorter (e.g., radius less than 13.5 mm and/or arc less than 106°). Some possible examples of the needle may have a curve with an arc of about 80°-125° and/or a radius of about 11 mm-16 mm.

In the example shown, there are 20 cuts. However, the number of cuts 408 can vary, with the variation of laser cuts affecting the curvature of the curved needle end 404. For example, some examples may have less than 10 cuts, 10-20 cuts, more than 20 cuts, at least 10 cuts, etc. Adding more cuts increases the curvature while reducing the number of cuts reduces the curvature. As shown, the cuts extends to about halfway through the profile of the needle and may be about 1 mm or less wide. However, the depth and/or width of the cuts can vary depending on the desired characteristics for the needle. The cuts may be spaced a few millimeters apart. In some examples, the cuts are uniform. The cuts may be aligned in parallel along a same side or surface of the needle end 404, with the same side or surface forming the inner curve of the curved needle end 404. The cuts may be made via laser or mechanically by a blade or other cutting mechanism.

The cuts may expose the lumen 406 formed by the walls of the needle. In a sample operation, guidewires would travel along the interior surface of the lumen wall away from the cuts, so would not catch on the cuts. However, in some examples, a sheath, sealant, or other covering may be placed over the cuts to maintain the integrity of the lumen while still allowing the needle to flexibly curve. Some examples of the implant delivery device may be configured to detect changes in pressure, so using the sheath can make the pressure within the lumen more consistent by reducing the ingress and egress of fluid into the lumen 406.

In the illustrated example, the tip of the needle 400 is cut at an angle (e.g., about 25°) to create a sharpened tip. The sharpened tip can help the needle pierce through tissue walls. In one example manufacturing process, the needle tip is cut with a laser at the desired angle prior to shaping of the needle. The cuts 408 may then be made by the laser to provide the desired curvature for the needle.

FIGS. 4D-4F show the needle 400 transitioning from a first configuration to a second configuration as the needle leaves a catheter 420. In the illustrated example, the catheter 420 has two lumens 422, 424, with the needle 400 in the first lumen 422. The first lumen 422 includes an opening 428 to the outside in the outer wall 426 of the catheter 420. In the first configuration shown in FIG. 4D, the needle 400 is in a generally straight configuration. However, there may be one or more slight bends in the needle to facilitate moving the needle through the catheter 420. While the needle end 404 is configured to curve, the needle end 404 material is flexible enough that the walls of the catheter lumen 422 are enough to keep the needle end 404 relatively straight. Relatively straight may mean there are one or more slight bends in the needle end 404 of a few degrees, but the needle end 404 remains straight enough that the needle 400 can be advanced through the catheter. Meanwhile, the needle body 402 may bend by even more, as the needle body 502 winds its way to the heart along various bodily lumens. One way of measuring the difference between the first configuration and the second configuration is by measuring the change in the direction pointed to by the needle tip, which may be a change of several tens of degrees. For example, if in the first configuration, the needle tip is pointing forward (about 180° as reflected in FIG. 4D), then in the second configuration the needle tip is pointing to the upper right (about 75° as reflected in FIG. 4F), a change in the needle tip direction of about 105°. In some examples, the direction of the needle tip changes by at least 900 when the needle end 404 transforms from the first configuration to the second configuration.

As the needle end 404 approaches the opening 428, the natural bias of the needle end 404 to curve can cause the needle end 404 to begin exiting the catheter 420, as shown in FIG. 4E. As the needle 400 is further advanced through the catheter 420, the entire needle end 404 exits the catheter 420, allowing the needle end 404 to enter its curved, second configuration, as shown in FIG. 4F. Another way of measuring the difference between the first configuration and the second configuration is by drawing a line from the tip to the needle to the deflection point where the needle end 404 bends from the needle body and measuring the angle created. In the illustrated example of FIG. 4F, the tip of the needle end 404 relative to the needle body 402 forms an angle of roughly 110 degrees. Some examples may form an angle of around 90-135 degrees. Some examples have an angle of less than 90 degrees, such that the needle end 404 forms, U-shape or hook, with the end pointing towards the proximal end.

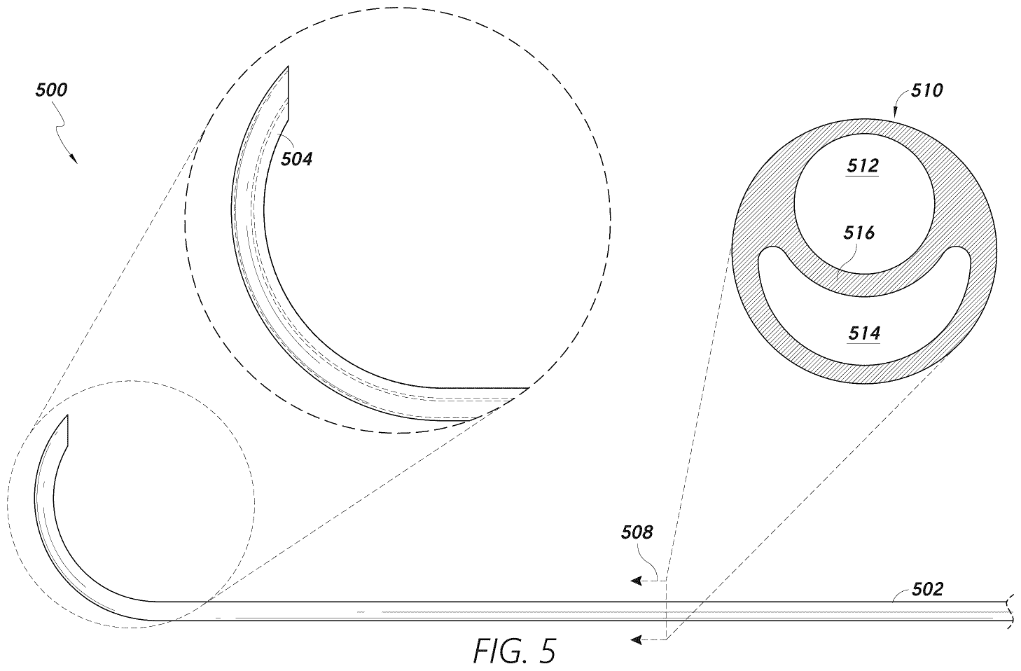

FIG. 5 illustrates another example of the curved needle 500 with a different structure and material, in accordance with one or more examples. In the illustrated example, at least a portion of the needle 500 is formed using a pliable material, such as nylon or a similar polymer. Materials like nylon that have excellent shape retention due to good elastic recovery behavior can be used for forming the needle. In some examples, the needle end 504 is formed of nylon or the like while the needle body 502 is formed of another material, such as steel.

The needle 500 of FIG. 5 can operate similarly to the needle 400 in FIGS. 4D-4F, where the needle end 504 is in a first configuration while in a catheter, but in a second configuration when exiting the catheter. In the first configuration, the interior catheter walls keep the needle end 504 in a relatively straight position relative to the needle body 502, though there may be slight curves of several degrees in the needle end. In addition, the needle body 502 may bend by even more, as the needle body 502 winds its way to the heart along various bodily lumens. However, the needle end 504 and needle body 502 can be considered in alignment, with both sections pointing in the same direction (e.g., distal end of needle end 504 and distal end of needle body 502), toward the target tissue location. In the second configuration, the needle end 504 returns to its natural shape, which is the curved shape shown in FIG. 5. Similar to the needle 400 in FIGS. 4D-4F, the nylon needle 500 is hollow, allowing a guidewire to be delivered to a target site within the needle 500.

A cross-cut 510 of the needle 500 taken at line 508 shows the interior structure of the needle 500, according to certain examples. In the illustrated example, the needle 500 includes a first lumen 512 and a second lumen 514. A lumen wall 516 can extend from one side of the outer wall to another side of the outer wall, dividing the first lumen from the second lumen. The wall may be straight or curved, depending on the desired shape of the lumens. The lumen wall 516 may provide additional stiffness to the needle by bracing the outer walls of the needle.

As shown in the cross-cut 510, the first lumen 512 is round while the second lumen 514 is crescent shape. Other examples may use different lumen shapes, depending on the desired properties for the needle. For example, a round shape lumen may be shaped for a guidewire. Using a crescent shape lumen that partially surrounds the round shape lumen 512 can increase the space available within the needle 500 for the lumens.

As will be apparent, other examples of the needle 500 may have a different number of lumens or different shaped lumens. For example, one example may have a single lumen, while other examples may have three or even more lumens. The above cross-cut 510 shows a round needle body, but other needles may use different shapes. For example, cutting needles are triangular in shape, and have 3 cutting edges to penetrate tough tissue. Reverse cutting needles have a cutting surface on the convex edge, and are ideal for tough tissue such as tendon or subcuticular suture. As those types of needles do not have a round shape, other lumen shapes (e.g., triangular) may make more efficient use of the space within those needle bodies.

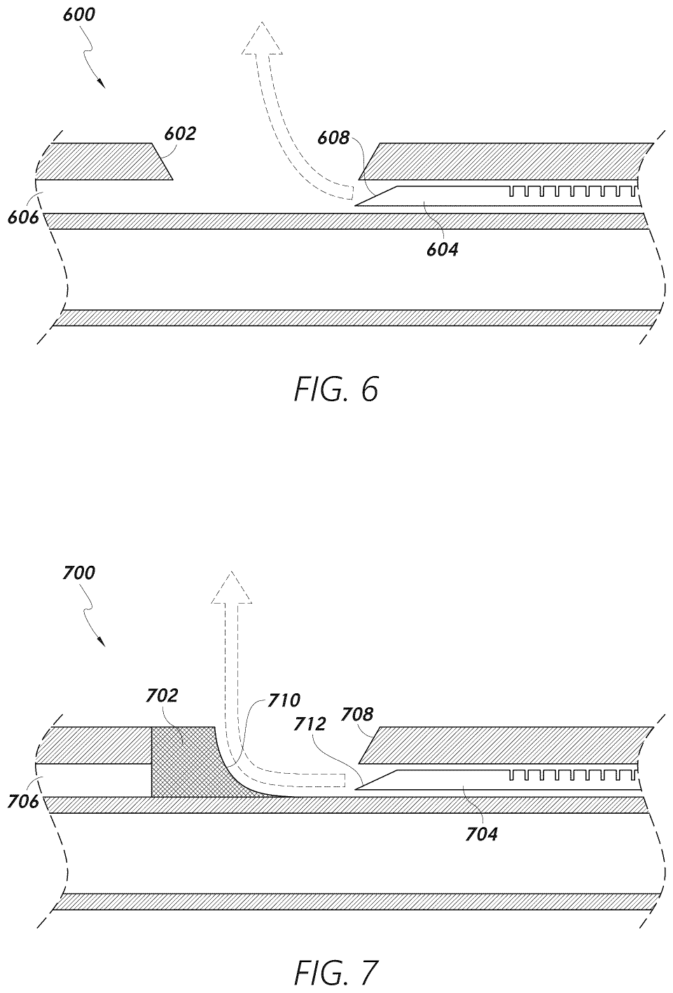

FIGS. 6 and 7 show different examples of a catheter for an implant delivery device, in accordance with one or more examples. In FIG. 6, the catheter 600 functions similarly to the catheter of FIG. 4D-4F, where the catheter has an opening 602 in outer wall. The opening 602 allows a needle 604 to exit its lumen 606 in the catheter. This type of catheter 600 can work well with a needle 604 that is biased to curve towards the opening 602. As the needle 604 passes the opening, its natural tendency to bend forces the end 608 of the needle out of the catheter. The needle then leaves the catheter.

FIG. 7 illustrates a catheter 700 with a ramp 702, according to certain examples. As the needle 704 advances through its lumen 706, it approaches an opening 708 and the ramp 702. In some examples, the ramp 702 closes off the distal end of the lumen 706. In the illustrated example, The curved surface 710 of the ramp forms part of the wall of the opening 708. As the needle 704 advances along down the lumen and along the curved surface 710, the distal end of the needle 712 is directed through the opening and outside of the catheter 700, along the direction shown by the arrow.

Using the ramp 702 to deflect the needle 704 can allow for needle designs that do not naturally curve. Rather, as long as the needle 704 is pliable, the ramp 702 can cause the needle 704 to deflect, redirecting the needle 704 out of the lumen 706. Thus, in some examples, the catheter 700 can be combined with a hollow needle 704 that may be straight when removed from the catheter 700. Rather, the ramp 702 is what causes the needle 704 to bend, rather than the needle 704 itself inherently curving when unconstrained by the walls of the catheter lumen 706. In a first configuration prior to hitting the ramp, the needle 704 remains relatively straight. In a second configuration after hitting the ramp the needle 704 becomes bent or curved due to the ramp.

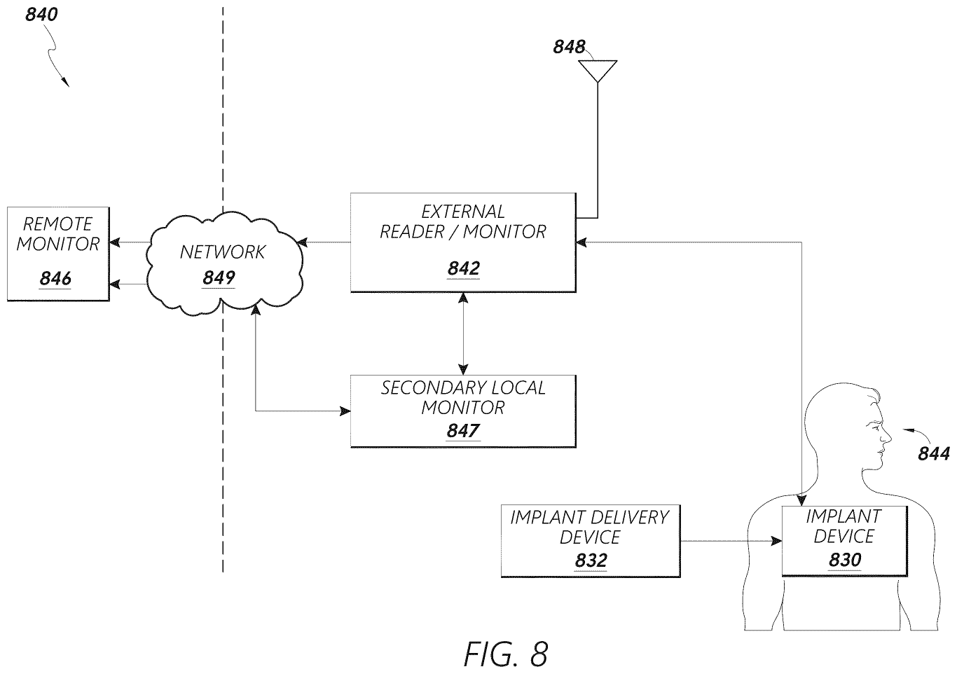

FIG. 8 shows a block diagram of a system 840 for implanting and/or monitoring an implant device in a patient 844 according to one or more examples. The system may monitor one or more physiological parameters (e.g., left atrial pressure and/or volume). The patient 844 can have a medical implant device 830 implanted in, for example, the heart (not shown), or associated physiology, of the patient 844 using an implant delivery device 835, such as those described in earlier figures. For example, the implant device 830 can be implanted at least partially within the left atrium and/or coronary sinus of the patient's heart. The implant device 830 may be a shunt implant device or other type of implant, such as devices associated with a sensor device that facilitate monitoring. For example, the implant device 830 can include one or more sensor transducers, such as one or more microelectromechanical system (MEMS) devices (e.g., MEMS pressure sensors, or other type of sensor transducer).

The implant device 830 may be installed using an implant delivery device 832. The implant delivery device 832 can include a catheter, a handle, and a curved needle. Examples of the curved needle are shown in FIGS. 4-7. The curved needle can be used to facilitate delivery of the implant device 830 to the target location (e.g., in the heart).

In certain examples, the system 840 provide monitoring functionality using at least two subsystems, including an implantable internal subsystem or device 830. The implant device 830 can include control circuitry comprising one or more microcontroller(s), discrete electronic component(s), and one or more power and/or data transmitter(s) (e.g., antennae coil). The system 840 can further include an external (e.g., non-implantable) subsystem that includes an external reader 842 (e.g., coil), which may include a wireless transceiver 848 that is electrically and/or communicatively coupled to certain control circuitry. In certain examples, both the internal 830 and external 842 subsystems include one or more corresponding coil antennas for wireless communication and/or power delivery through patient tissue disposed therebetween. The sensor implant device 830 can be any type of implant device. For example, in some examples, the implant device 30 comprises a pressure sensor integrated with another functional implant structure 39, such as a prosthetic shunt or stent device/structure.

In certain examples, the implant device 830 can be configured to generate electrical signals that can be wirelessly transmitted to a device outside the patient's body, such as the illustrated local external monitor system 842. In order to perform such wireless data transmission, the implant device 830 can include radio frequency (RF) (or other frequency band) transmission circuitry, such as signal processing circuitry and an antenna. The antenna can comprise an antenna coil implanted within the patient. The control circuitry may comprise any type of transceiver circuitry configured to transmit an electromagnetic signal, wherein the signal can be radiated by the antenna, which may comprise one or more conductive wires, coils, plates, or the like. The control circuitry of the implant device 830 can comprise, for example, one or more chips or dies configured to perform some amount of processing on signals generated and/or transmitted using the device 830. However, due to size, cost, and/or other constraints, the implant device 830 may not include independent processing capability in some examples.

The wireless signals generated by the implant device 830 can be received by the local external monitor device or subsystem 842, which can include a reader/antenna-interface circuitry module configured to receive the wireless signal transmissions from the implant device 830, which is disposed at least partially within the patient 44. For example, the circuitry module may include transceiver device(s)/circuitry.

The external local monitor 842 can receive the wireless signal transmissions from the implant device 830 and/or provide wireless power to the implant device 830 using an external antenna, such as a wand device. In some examples, the local monitor device 842 can serve as an intermediate communication device between the implant device 830 and the remote monitor 846. The local monitor device 842 can be a dedicated external unit designed to communicate with the implant device 830. For example, the local monitor device 842 can be a wearable communication device, or other device that can be readily disposed in proximity to the patient 844 and implant device 830. The local monitor device 842 can be configured to interrogate the implant device 830 continuously, periodically, or sporadically in order to extract or request sensor-based information therefrom. In certain examples, the local monitor 842 comprises a user interface, wherein a user can utilize the interface to view sensor data, request sensor data, or otherwise interact with the local monitor system 842 and/or implant device 830.

The system 840 can include a secondary local monitor 847, which can be, for example, a desktop computer or other computing device configured to provide a monitoring station or interface for viewing and/or interacting with the monitored cardiac pressure data. In an example, the local monitor 842 can be a wearable device or other device or system configured to be disposed in close physical proximity to the patient and/or implant device 830, wherein the local monitor 842 is primarily designed to receive/transmit signals to and/or from the implant device 830 and provide such signals to the secondary local monitor 847 for viewing, processing, and/or manipulation thereof. The external local monitor system 842 can be configured to receive and/or process certain metadata from or associated with the implant device 830, such as device ID or the like, which can also be provided over the data coupling from the implant device 830.

The remote monitor subsystem 846 can be any type of computing device or collection of computing devices configured to receive, process and/or present monitor data received over the network 849 from the local monitor device 842, secondary local monitor 847, and/or implant device 830. For example, the remote monitor subsystem 846 can advantageously be operated and/or controlled by a healthcare entity, such as a hospital, doctor, or other care entity associated with the patient 844. Although certain examples disclosed herein describe communication with the remote monitor subsystem 846 from the implant device indirectly through the local monitor device 842, in certain examples, the implant device 830 can comprise a transmitter capable of communicating over the network 849 with the remote monitor subsystem 846 without the necessity of relaying information through the local monitor device 842.

While the above has described the implant delivery device 832 as part of a monitoring system, the implant delivery device 832, including the curved needle examples described in previous figures, can be utilized with non-monitoring implants, such as shunts, balloons, valves or the like. In addition, the implant delivery device 832 may be used with manual medical devices and does not necessarily need to be used with computerized medical devices and systems.

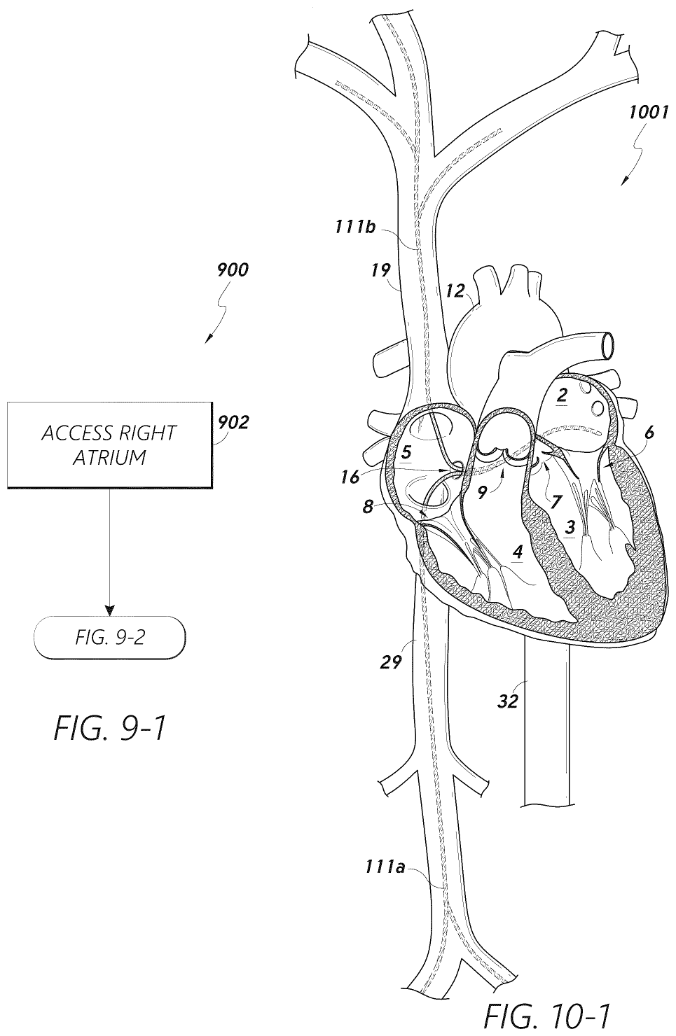

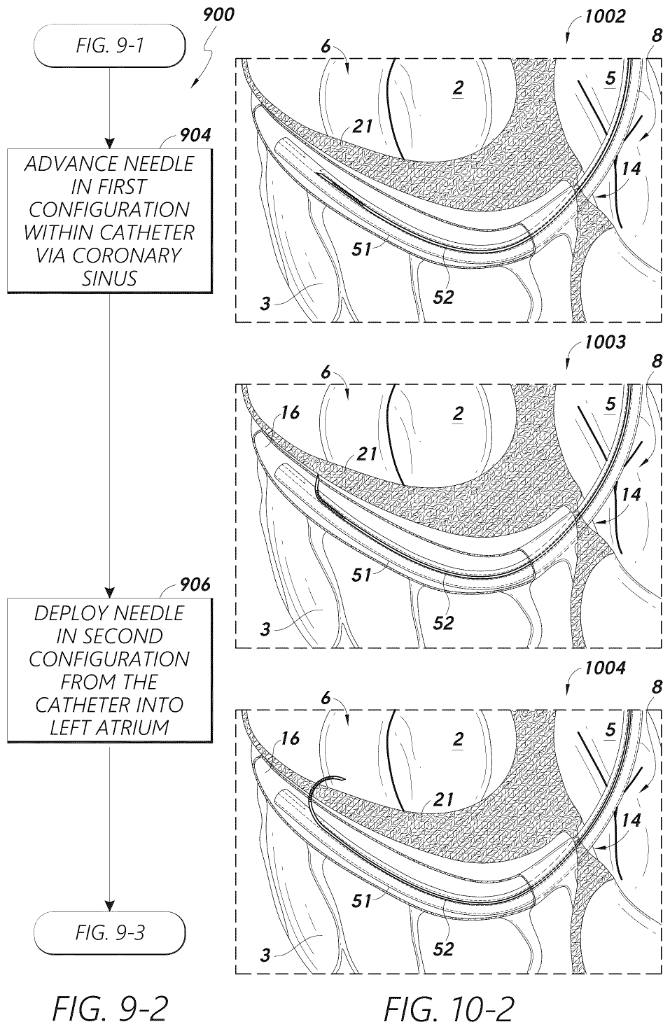

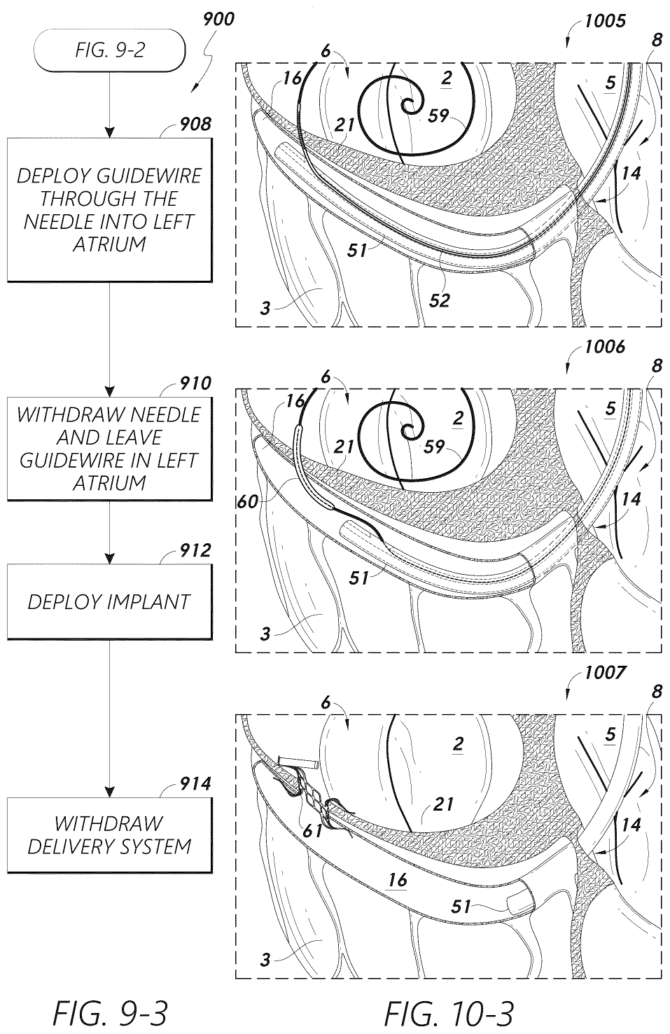

FIGS. 9-1, 9-2, and 9-3 (collectively referred to as FIG. 9), provides a flow diagram representing a process 900 for using an implant delivery device that uses a curved needle to perform a medical procedure, according to one or more examples disclosed herein. A health provider, such as a surgeon, can use the process 900 during a transseptal, trans aorta, transfemoral artery, trans radial, transapical, or other surgical approach to perform a medical procedure (e.g., installing a stent, valve, or other implant). In addition, for ease of explanation, the following uses the label numbering from prior figures in referring to certain structures or components. However, the process is not limited to the specific example illustrated in those figures. For example, the example of FIGS. 4A-4F, FIGS. 5-7, as well as other examples of the anchor placement device can perform the process 900.

Corresponding FIGS. 10-1, 10-2, and 10-3 (collectively referred to as FIG. 10) provide a view of the implant delivery device in the heart as process 900 is performed.

At block 902, the process 900 involves accessing a right atrium 5 of a heart of a patient with a catheter-based implant delivery device 111 (labeled as 111a and 111b in FIG. 10). Image 1001 of FIG. 10-1 shows various catheter-/sheath-type delivery devices/systems 111 that may be used to implant devices in accordance with aspects of the present disclosure. The implant delivery device 111 can advantageously be steerable and relatively small in cross-sectional profile to allow for traversal of the various blood vessels and chambers through which they may be advanced en route to, for example, the right atrium 5, coronary sinus 16, left atrium 2 or other anatomy or chamber. Catheter access to the right atrium 5, coronary sinus 16, or left atrium 2 in accordance with certain transcatheter solutions may be made via the inferior vena cava 29 (as shown by the catheter 111a) or the superior vena cava 19 (as shown by the catheter 111b).

Although access to the right and/or left atria is illustrated and described in connection with certain examples as being via the right atrium and/or vena cavae, such as through a transfemoral or other transcatheter procedure, other access paths/methods may be implemented in accordance with examples of the present disclosure. For example, in cases in which septal crossing through the interatrial septal wall is not possible, other access routes may be taken to the left atrium 2. In patients suffering from a weakened and/or damaged atrial septum, further engagement with the septal wall can be undesirable and result in further damage to the patient. Furthermore, in some patients, the septal wall may be occupied with one or more implant devices or other treatments, wherein it is not tenable to traverse the septal wall in view of such treatment(s). As alternatives to transseptal access, transaortic access may be implemented, wherein a delivery catheter is passed through the descending aorta 32, aortic arch 12, ascending aorta, and aortic valve 7, and into the left atrium 2 through the mitral valve 6. Alternatively, transapical access may be implemented to access the target anatomy.

The process 900 may involve placing a guidewire 59 in the left atrium 2 via a pathway through the coronary sinus. For example, such guidewire placement may involve one or more of the operations associated with blocks 908 and 910, which, as with any other of the blocks of the process 900, may be considered optional operations. The operations associated with blocks 904 and 906 relate steps in making a puncture hole through a wall of the coronary sinus for advancing the guidewire 59 into the left atrium.

Any of several access pathways visible and/or apparent in the image 1001 may be implemented for maneuvering guidewires and delivery systems/catheters in and around the heart to implement any of the operations associated with the various blocks of the process 900. For instance, access may be from above via either the subclavian or jugular veins into the superior vena cava 19, right atrium 5 and from there into the coronary sinus 16. Alternatively, the access path may start in the femoral vein and through the inferior vena cava 29 into the heart. Other access routes may also be used, and each typically utilizes a percutaneous incision through which a guidewire and/or catheter are inserted into the vasculature, normally through a sealed introducer, and from there the physician controls the distal ends of the devices from outside the body.

At block 904, the process 900 involves advancing the catheter 51 via the coronary sinus 16. Within the catheter is, in accordance with some examples, a hollow, curving needle 52, such as those described in FIGS. 4-7. As discussed above, the needle can be in at least two configurations. In the first configuration, the needle is relatively straight in the catheter 51. The walls of the catheter can constrict the needle and prevent the needle from curving.

The catheter 51 can be introduced into the body through a proximal end of an introducer sheath (not shown). The introducer sheath can be configured to provide access to the particular vascular pathway (e.g., jugular or subclavian vein) utilized, and may have one or more hemostatic valves associated therewith/therein. While holding the relevant introducer sheath at a fixed location, the surgeon can manipulate the catheter 51 to the implant site. One or more radiopaque markers may be disposed on the catheter 51 to help the surgeon determine the precise advancement distance for the catheter within the coronary sinus.

Since the coronary sinus 16 is largely contiguous around the left atrium 2, there are a variety of possible acceptable placements for an implant or other medical device. The site selected for placement of the implant (and, therefore, for puncture through the tissue wall 21), may be made in an area where the tissue of the particular patient is less thick or less dense, as determined beforehand by non-invasive diagnostic means, such as a CT scan or radiographic technique, such as fluoroscopy or intravascular coronary echo. Image 1002 of FIG. 10-2 shows the catheter 51 being advanced from the right atrium 5 into the coronary sinus 16 through its ostium or opening 14.

At block 906, the process 900 involves deploying the needle into its second configuration from the catheter into the left atrium 2, as seen in image 1003 and 1004. The needle 52 is configured to puncture the tissue wall 21 to gain access into the left atrium 2. Image 1003 shows the needle begin deployment from the catheter 51. As the end portion of the needle is biased to curve, tension is placed by the end portion of the needle against the interior wall of the catheter, which prevents the end portion from curving. As the distal end of the needle slides past an opening in the catheter wall, the tension against the wall is released, freeing the end portion of the needle to begin bending. This bending causes the distal end of the needle to angle upwards into the wall 21. As the needle 52 is advanced forward, the needle tip pierces into the wall. Advantageously, the curve in the needle can also orient the needle toward the left atrium 2 when deployed from the catheter 51.

At block 908, the process 900 involves deploying a guidewire 59 through the needle 52 into the left atrium 2. In some examples, the needle is hollow (e.g., a hypodermic needle), allowing a guidewire 59 to be pushed through the needle and into the left atrium 2 or into whatever target site the needle 52 is placed into. Image 1005 of FIG. 10-3 shows the guidewire 59 deploying from the needle 52.

At block 910, the process 900 involves withdrawing the needle 52 and leaving the guidewire 59 in the let atrium. Image 1006 shows the needle 52 withdrawn from the catheter 51 from the proximal end (e.g. the catheter handle). However, the needle 52 does not need to be completely withdrawn from the catheter. For example, if the catheter 51 will be moved to secondary target location, the needle 52 may remain within the catheter 51 after being withdrawn from the primary target location.

At block 912, the process 900 involves deploying an implant via the guidewire. For example, the implant may be a balloon 60, as shown in image 1006. In another example, the implant may be a shunt 61. Such a shunt implant device 61 may be deployed in the tissue wall 21 in order to shunt blood from the left atrium 2 into the coronary sinus 16. Image 1007 shows a shunt being deployed from the guidewire. In some situations, multiple medical devices may be used. For example, the balloon 60 may be used to enlarge the opening so that the shunt 61 may be deployed. While the above describes a balloon and a shunt, other types of medical implants or devices may be used with the implant delivery system.

At block 914, the process 900 involves withdrawing the delivery system, including the catheter 51, from the deployment site. Included in such withdrawal is the guidewire 59, which is also removed from the patient's anatomy. Image 1007 shows the catheter 51 being withdrawn from the coronary sinus 16.

Although described in the context of implanting a device in the wall between the coronary sinus and left atrium, it should be understood that the process 900 may be implemented, at least in part, to implant or deliver a medical device in other anatomy and/or tissue walls, such as the atrial septum or ventricular septum.

FIG. 11 illustrates various examples of needle ends (also called needle points) that may be used with the examples of the hollow needle described in the earlier figures. Needle ends may be formed in various shapes that are appropriate for different uses. For example, taper-point needles provide good penetration of soft tissue. Blunt taper points have blunder ends to reduce the risk of needle stick injury. The needle ends may be straight, curved, or angled depending on the desired usage.

Needle end 1102 includes a needle edge at a relatively shallow angle. The needle edge is formed from one side of the outer needle wall to the other, opposite side of the outer needle wall. For example, the angle formed by the needle edge at the point of the needle may be around 40-50 degrees.

Needle end 1104 includes a needle edge at a sharply steeper angle than needle end 1102. For example, the angle formed at the point of the needle may be around 10-20 degrees. Needle end 1106 includes a needle edge forming an angle of around 30-40 degrees. Needle end 1108 includes a needle edge forming an angle of around a 20-30 degree at the point of the needle.

Needle end 1110 includes an angled end, with a needle edge formed relatively parallel to a needle body section closest to the needle end 1110. The angled needle end can facilitate a change in direction as a guidewire leaves the needle.

The above are just some examples of needle ends that can be used with the various needles of implant delivery devices described herein. Depending on the desired use case such as the entry point or route used through the body, various types of needle ends may be more beneficial to use.

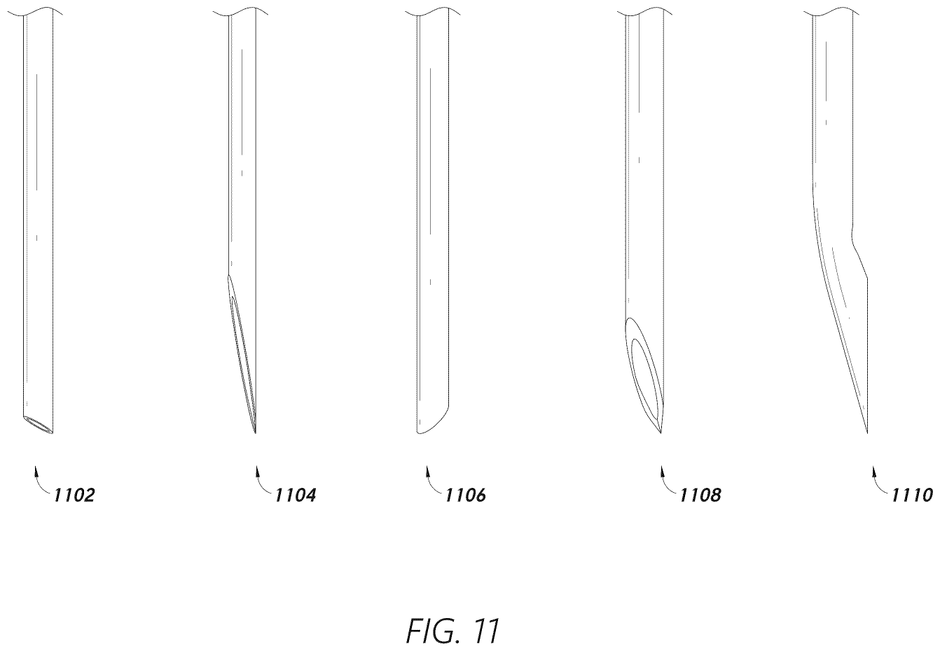

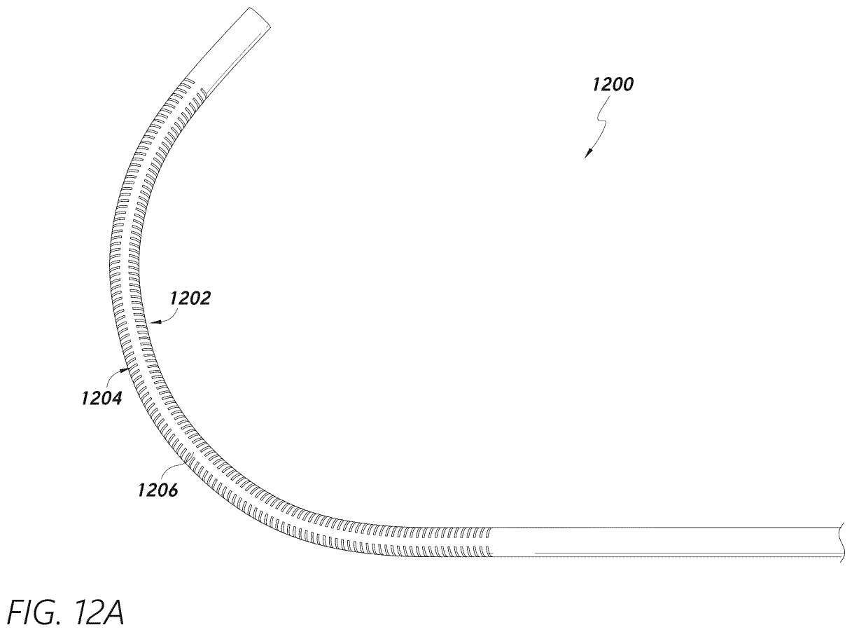

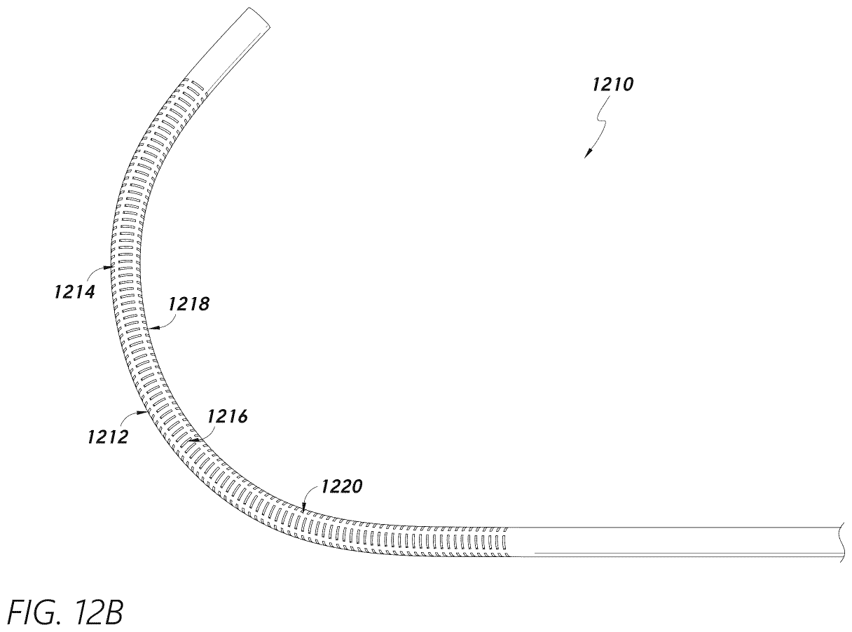

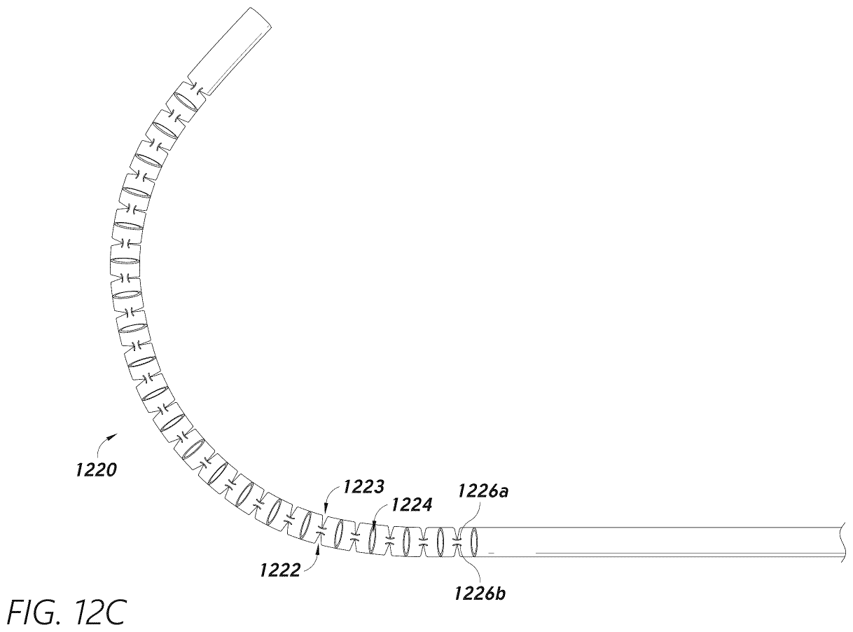

FIGS. 12A-12C illustrate various cut patterns that can be used on the needle body of examples of the hollow needle described above. FIG. 12A illustrates a cut pattern with at least 100 cuts. As discussed above, the number of cuts in the pattern can vary, with the variation of cuts (e.g., made using a laser or other cutting tool) affecting the curvature of the curved needle end. In comparison to the cuts in FIGS. 4A-4C, the cut pattern in FIGS. 12A-12C may be thinner and closer together, the same size and spread over a larger surface area of the needle, and/or both thinner and covering a larger surface area of the needle. Generally, adding more cuts increases the curvature while reducing the number of cuts reduces the curvature of the needle. The cuts may have a width of about 1 mm, 0.8 mm, 0.5 mm, or even narrower. However, the depth and/or width of the cuts can vary depending on the desired characteristics for the needle. The cuts may be spaced a few millimeters or less apart. In some examples, the cuts are uniform. Other examples may have varying cuts. The cuts may be aligned in parallel along a same side or surface of the needle end. The cuts may be a single column or multiple columns, for example, as shown in FIGS. 12A-12B. The cuts may also be in more complex patterns, for example, as shown in FIG. 12C.

FIG. 12A illustrates a needle 1200 with two columns of cuts 1202, 1206. The cuts are separated by a spine 1204 on one side of the needle, with another spine (not shown) on the opposite side. The spines 1204 provide rigidity and support to the needle 1200, while the cuts provide flexibility. For example, in a layout with spines on the top and bottom, the spines inhibit up and down movement while the cuts allow left and right movement of the needle 1200. As will be apparent, the needle may be rotated to face the cuts in the direction (e.g., up/down, side-to-side) where flexibility is desired.

FIG. 12B illustrates a needle 1210 with three columns of cuts 1212, 1216, 1218. Between the first column 1212 and the second column 1216 is a first spine 1214. Between the second column 1216 and the third column 1218 is a second spine 1218. Between the third column 1218 and the first column 1212 is a third spine (not shown). By having three columns of cuts, flexibly is provided in three different directions (e.g., at 120 degrees, at 240 degrees, and at 360 degrees relative to the needle's outer diameter). Meanwhile, the needle remains stiffer along the spines.

FIG. 12C illustrates a needle 1220 having an alternating pattern of cuts. A first set of cuts are made in a first column, with a second set of cuts opposite the first set of cuts in a second column. Interleaved with the first and second set of cuts are a third set of cuts rotated 90 degrees relative to the first set of cuts and a fourth set of cuts opposite the third set of cuts. For example, cut 1224 has an opposite cut (not shown) on the other side of the needle. Cut 1222 (shown in a side profile) has an opposite cut 1223 on the other side of the needle.

In some examples, the corners of the cuts are scored to facilitate bending at the corners of the cuts. For example, a first cut has a first scoring 1226a at one of its corners while the a second cut opposite the first cut has a second scoring 1226b at one of its corners. The first and second cuts have a corresponding scorings on the their other corners (not shown).

As will be apparent, the above are just some examples of cut patterns. Other cut patterns can be used to provide flexibility and stiffness along the desired directions. For example, one or more columns of cuts can be used to provide flexibility while one or more spines can be formed along the needle body to provide stiffness.

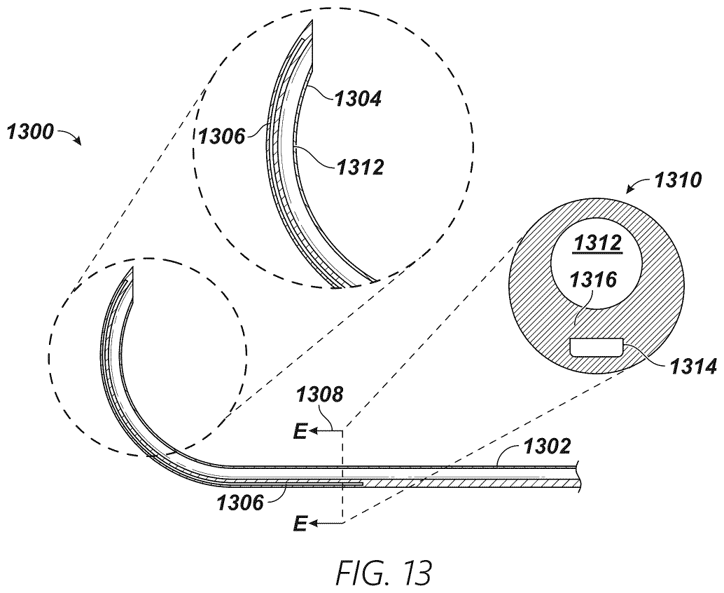

FIG. 13 illustrates a needle 1300 that uses a wire stiffener to maintain its shape, in accordance with one or more examples. The needle 1300 can be made of a plastic material, such as polycarbonate, polypropylene, acrylonitrile butadiene styrene (ABS), polyethylene terephthalate glycol (PETG), polyethylene, polymethyl methacrylate, polyvinyl chloride (PVC), and/or nylon. While plastic materials are pliable, such materials may not have enough elasticity to fully restore itself to the intended curvature of the needle 1300. By using a wire stiffener 1306, using a metal material with better elasticity than the plastic, the overall elasticity of the needle 1300 can be improved, enabling the curvature of the needle 1300 to be restored after the needle 1300 leaves an introducer sheath or catheter.

The needle 1300 can operate similarly to the needle 400 in FIGS. 4D-4F, where the needle end 1304 is in a first configuration while in a catheter, but in a second configuration when exiting the catheter. In the first configuration, the interior catheter walls can keep the needle end 1304 in a relatively straight position relative to the needle body 1302, though there may be slight curves of several degrees in the needle end. In addition, the needle body 1302 may bend by even more, as the needle body 1302 winds its way to the heart along various bodily lumens. However, the needle end 1304 and needle body 1302 can be considered in alignment, with both sections pointing in the same direction (e.g., distal end of needle end 1304 and distal end of needle body 1302), toward the target tissue location. In the second configuration, the needle end 1304 returns to its natural or intended shape, which is the curved shape shown in FIG. 13. Similar to the needle 400 in FIGS. 4D-4F, the needle 1300 is hollow, allowing a guidewire to be delivered to a target site within the needle 500.

A cross-cut 1310 of the needle 1300 taken at line 1308 shows the interior structure of the needle 1300, according to certain examples. In the illustrated example, the needle 1300 includes a first lumen 1312 and a second lumen 1314. A lumen wall 1316 can extend from one side of the outer wall to another side of the outer wall, dividing the first lumen from the second lumen. The wall may be straight or curved, depending on the desired shape of the lumens. The lumen wall 1316 may provide additional stiffness to the needle by bracing the outer walls of the needle. The wire stiffener 1306 may be configured to fit into the second lumen 1314. Meanwhile, the first lumen 1312 can be used to deliver a guidewire or other device to a target tissue site.

As shown in the cross-cut 1310, the first lumen 1312 has a round cross-section while the second lumen 1314 has a generally rectangular cross-section. Other examples may use different lumen shapes or dimensions, depending on the desired properties for the needle. For example, the second lumen 1314 may be enlarged to fit a thicker wire stiffener 1306, if greater stiffness and/or elasticity is desired.

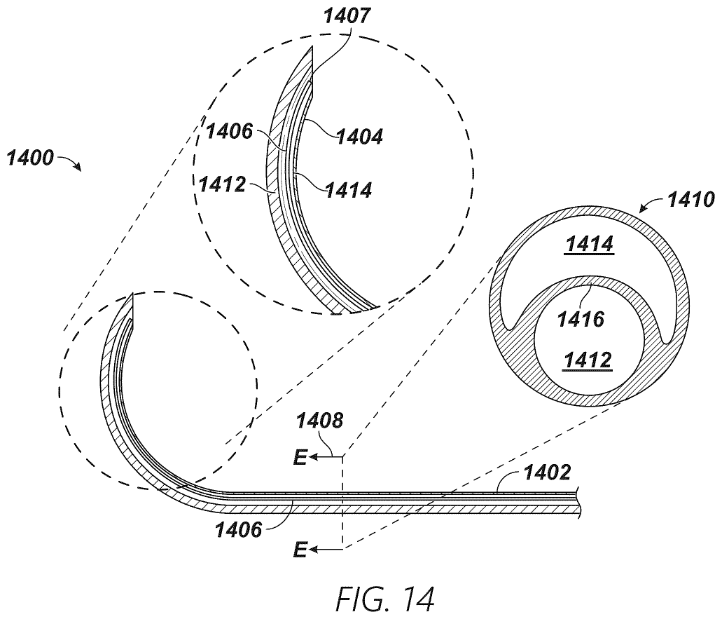

FIG. 14 illustrates a needle 1400 that uses a wire stiffener 1406 along its interior curvature to maintain its shape, in accordance with one or more examples. The needle 1400 of FIG. 14 is similar in functionality to the needle 1300 of FIG. 13, differentiated mainly in the location of the wire stiffener 1406. Placing the wire stiffener 1406 along the interior curvature may facilitate better force transfer from the wire stiffener 1406 to the needle body 1402 and needle end 1404, as the wire stiffener 1406 is in contact with the interior curving surface of the needle 1400.

Similar to the needle of FIG. 13, the needle 1400 can be made of a plastic material, such as nylon or a similar polymer. The wire stiffener 1406 can use a metal material to improve the overall elasticity of the needle 1400. The needle 1400 can operate similarly to the needle 400 in FIGS. 4D-4F, where the needle end 1404 is in a first configuration while in a catheter, but in a second configuration when exiting the catheter. Similar to the needle 400 in FIGS. 4D-4F, the needle 1400 is hollow, allowing a guidewire to be delivered to a target site within the needle 1400.

Needles 1400 made of plastic material may not have enough rigidity to pierce through tissue. In those examples, the wire stiffener 1406 can include a needle tip 1407. In some implementations, the wire stiffener 1406 may be used to pierce through tissue, to create an opening for the needle. In one example implementing, the wire stiffener 1406 can be pushed out of the hollow needle such that the needle tip of the wire stiffener 1406 extends past the needle end 1404. The extended needle tip 1407 can then be used to pierce through tissue, creating an opening for the needle 1400.

A cross-cut 1410 of the needle 1400 taken at line 1408 shows the interior structure of the needle 1400, according to certain examples. In the illustrated example, the needle 1400 includes a first lumen 1412 and a second lumen 1414. A lumen wall 1416 can extend from one side of the outer wall to another side of the outer wall, dividing the first lumen from the second lumen. The wall may be straight or curved, depending on the desired shape of the lumens. The lumen wall 1416 may provide additional stiffness to the needle by bracing the outer walls of the needle. The wire stiffener 1406 may be configured to fit into the second lumen 1414. Meanwhile, the first lumen 1414 can be used to deliver a guidewire or other device to a target tissue site.

As shown in the cross-cut 1410, the first lumen 1412 has a round cross-section while the second lumen 1414 has a crescent-shaped cross-section. Other examples may use different lumen shapes or dimensions, such as shown in FIG. 13, depending on the desired properties for the needle. For example, the second lumen 1414 may be enlarged to fit a thicker wire stiffener 1406, if greater stiffness and/or elasticity is desired.

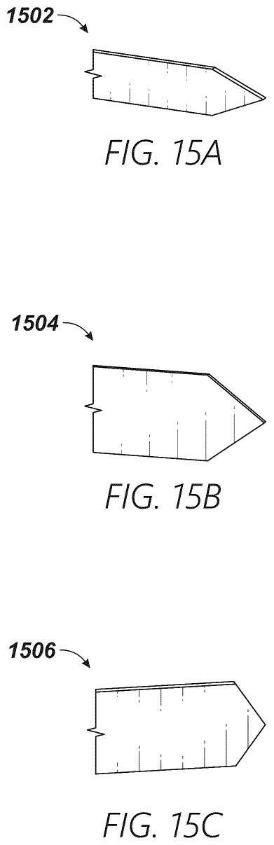

FIGS. 15A-15C illustrate various examples of wires stiffeners, in accordance with one or more examples. Wire stiffener may include needle tips, which may be of varying design, to aid in piercing tissue site. The needle tips may be formed on the wire stiffeners using a diamond grind, laser cut, buffing, and/or other manufacturing process. The stiffeners may be sized to a desired length, based on the size of the hollow needles. In some examples, the wire stiffeners are relatively short, at around 15 centimeters.

The first wire stiffener 1502 is illustrated as a narrow strip, with a pointed end of about 30-45 degrees, measured from slanted edge to slanted edge. As shown, the thickness of the wire stiffener 1502 is a fairly thin, which can enable the wire stiffener to bend more easily. The second wire stiffener 1504 is illustrated as a relatively wider strip, with a pointed end of about a 45-80 degrees. The third wire stiffener 1506 is illustrated as a relatively wider strip compared to the first wire stiffener 1502, with a pointed end of about 90-110 degrees. As will be apparent, the wire stiffeners may be made in various widths and with various needle tips, based on the desired performance of the wire stiffeners.

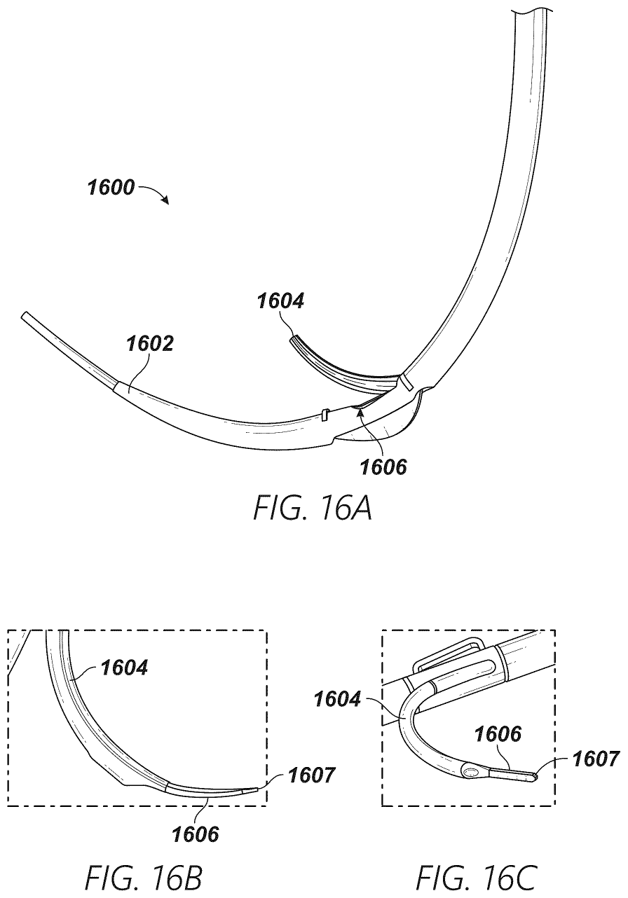

FIGS. 16A-16C illustrate various views of a device 1600 comprising a needle 1604 that uses a wire stiffener 1606, and a sheath 1602, according to one or more examples disclosed herein. FIG. 16A illustrates a side view of the sheath 1602, having an opening 1606 for deploying the needle 1604. As the needle 1604 is extended out of the sheath, the needle 1604 is restored to its intended curvature. FIG. 16B is a close-up view of the needle 1604 from an opposite side as FIG. 16A. In the illustrated view, the wire extender 1606 is shown extended past the distal end of the needle 1604. The tip 1607 of the wire extender 1606 can be used to pierce through tissue. FIG. 16C is a perspective close-up view of the needle 1604 showing the wire extender 1606 extended past the distal end of the needle 1604.

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below.

Example 1: A device for delivering a guidewire to a target location in a patient's body, the device comprising: a hollow needle comprising: a lumen configured to receive the guidewire; a body section of the hollow needle; and a biased section of the hollow needle, the biased section configured to bend relative to the body section, the biased section located on a distal end of the hollow needle; and a catheter comprising a catheter lumen, the catheter lumen configured to receive the hollow needle and maintain the biased section and the body section in relative alignment in a first configuration, wherein the hollow needle, in response to the biased section extending out from the catheter, is configured to bend the biased section relative to the body section in a second configuration.

Example 2: The device of any example herein, in particular example 1, and further comprising a plurality of cuts made in the biased section, the plurality of cuts configured to allow the biased section to bend.

Example 3: The device of any example herein, in particular example 2, wherein the plurality of cuts are uniform and aligned in parallel along a same side of the biased section, the same side comprising an inner surface of a curve formed by the biased section.

Example 4: The device of any example herein, in particular any of examples 2 to 3, wherein the plurality of cuts number at least 10 cuts.

Example 5: The device of any example herein, in particular any of examples 2 to 4, wherein the plurality of cuts are laser cuts.

Example 6. The device of any example herein, in particular any of examples 2 to 5, further comprising a sealant over the plurality of cuts.

Example 7: The device of any example herein, in particular example 1, wherein the hollow needle further comprises: a second lumen configured to hold a wire stiffener, the wire stiffener; wherein the body section and the biased section are made from a plastic material and the wire stiffener is configured to maintain the relative bend of the biased section to the body section.

Example 8: The device of any example herein, in particular any of examples 1 to 7, the biased section further comprising a needle tip, wherein a direction pointed to by the needle tip changes by at least 90 degrees when transforming from the first configuration to the second configuration.

Example 9: The device of any example herein, in particular any of examples 1 to 8, the biased section having a radius of about 13.5 mm and an arc of about 106 degrees.

Example 10: The device of any example herein, in particular any of examples 1 to 9, wherein the biased section forms a U-shape in the second configuration.

Example 11: The device of any example herein, in particular any of examples 1 to 10, further comprising a ramp formed within the catheter lumen, the ramp configured to deflect the biased section of the hollow needle relative to the body section.

Example 12: The device of any example herein, in particular any of examples 1 to 11, wherein an angle formed by the body section and the biased section is no more than 90 degrees in the second configuration.

Example 13: The device of any example herein, in particular any of examples 1 to 12, wherein the catheter comprises an opening into the catheter lumen located along a side of the catheter, the opening configured to provide egress out of the catheter lumen for the hollow needle.

Example 14: A method for delivering a guidewire to a target location in a patient's body using a hollow needle deployed from a catheter, the method comprising: advancing the hollow needle in the catheter to a first location, the hollow needle comprising a biased section and a body section, the biased section and the body section maintained in relative alignment by the catheter in a first configuration; advancing the biased section of the hollow needle through an opening of the catheter; deploying the biased section of the hollow needle through the opening of the catheter into a second configuration, wherein in response to the biased section extending out from the catheter, the biased section is configured to bend relative to the body section; and deploying the guidewire from the hollow needle to the target location.

Example 15: The method of any example herein, in particular example 14, wherein deploying the biased section of the hollow needle comprises piercing through a tissue wall with the hollow needle to reach the target location.

Example 16: The method of any example herein, in particular example 15, further comprising deploying a medical implant to the target location using the guidewire.

Example 17: The method of any example herein, in particular any of examples 14 to 16, the biased section further comprising a needle tip, wherein a direction pointed to by the needle tip changes by at least 90 degrees when transforming from the first configuration to the second configuration.

Example 18: The method of any example herein, in particular any of examples 14 to 16, wherein the biased section forms a U-shape in the second configuration.

Example 19: The method of any example herein, in particular any of examples 14 to 16, further comprising after deploying the guidewire, withdrawing the hollow needle from the target location while retaining the hollow needle in the catheter.

Example 20: The method of any example herein, in particular any of examples 14 to 16, wherein the hollow needle is made at least partly from nylon or nitinol.

Example 21: A hollow needle for delivering a guidewire to a target location in a patient's body, the hollow needle comprising: a lumen configured to receive the guidewire; a body section of the hollow needle; and a biased section of the hollow needle located on a distal end of the hollow needle, the biased section comprising: a needle tip; and a plurality of cuts aligned in parallel along a same side of the biased section, the same side comprising an inner surface of a curve formed by the biased section, the plurality of cuts configured to cause the biased section to bend relative to the body section.

Example 22: The hollow needle of any example herein, in particular example 20, wherein the plurality of cuts comprises at least 10 cuts that are uniform.

Example 23: A hollow needle for delivering a guidewire to a target location in a patient's body, the hollow needle comprising: a lumen configured to receive the guidewire; a body section of the hollow needle; and a biased section of the hollow needle, the biased section configured to bend relative to the body section, the biased section located on a distal end of the hollow needle.

Example 24: The hollow needle of any example herein, in particular example 23, further comprising: a second lumen configured to hold a wire stiffener, the wire stiffener comprising a pointed end configured to pierce tissue; wherein the body section and the biased section are made from a plastic material and the wire stiffener is configured to maintain the relative bend of the biased section to the body section.

Example 25: The hollow needle of any example herein, in particular example 23, further comprising a plurality of cuts made in the biased section, the plurality of cuts configured to allow the biased section to bend.

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for case of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A device for delivering a guidewire to a target location in a patient's body, the device comprising:
a hollow needle comprising:
a lumen configured to receive the guidewire;
a body section of the hollow needle; and
a biased section of the hollow needle, the biased section configured to bend relative to the body section, the biased section located on a distal end of the hollow needle; and
a catheter comprising a catheter lumen, the catheter lumen configured to receive the hollow needle and maintain the biased section and the body section in relative alignment in a first configuration,
wherein the hollow needle, in response to the biased section extending out from the catheter, is configured to bend the biased section relative to the body section in a second configuration.

2. The device of claim 1, and further comprising a plurality of cuts made in the biased section, the plurality of cuts configured to allow the biased section to bend.

3. The device of claim 2, wherein the plurality of cuts are uniform and aligned in parallel along a same side of the biased section, the same side comprising an inner surface of a curve formed by the biased section.

4. The device of claim 2, wherein the plurality of cuts number at least 10 cuts.

5. The device of claim 2, further comprising a sealant over the plurality of cuts.

6. The device of claim 1, wherein the hollow needle further comprises:
a second lumen configured to hold a wire stiffener, the wire stiffener comprising a pointed end configured to pierce tissue;
wherein the body section and the biased section are made from a plastic material and the wire stiffener is configured to maintain the relative bend of the biased section to the body section.

7. The device of claim 1, the biased section further comprising a needle tip, wherein a direction pointed to by the needle tip changes by at least 90 degrees when transforming from the first configuration to the second configuration.

8. The device of claim 1, the biased section having a radius of about 13.5 mm and an arc of about 106 degrees.

9. The device of claim 1, wherein the biased section forms a U-shape in the second configuration.

10. The device of claim 1, further comprising a ramp formed within the catheter lumen, the ramp configured to deflect the biased section of the hollow needle relative to the body section.

11. The device of claim 1, wherein an angle formed by the body section and the biased section is no more than 90 degrees in the second configuration.

12. The device of claim 1, wherein the catheter comprises an opening into the catheter lumen located along a side of the catheter, the opening configured to provide egress out of the catheter lumen for the hollow needle.

13. A method for delivering a guidewire to a target location in a patient's body using a hollow needle deployed from a catheter, the method comprising:
advancing the hollow needle in the catheter to a first location, the hollow needle comprising a biased section and a body section, the biased section and the body section maintained in relative alignment by the catheter in a first configuration;
advancing the biased section of the hollow needle through an opening of the catheter;
deploying the biased section of the hollow needle through the opening of the catheter into a second configuration, wherein in response to the biased section extending out from the catheter, the biased section is configured to bend relative to the body section; and
deploying the guidewire from the hollow needle to the target location.

14. The method of claim 13, wherein deploying the biased section of the hollow needle comprises piercing through a tissue wall with the hollow needle to reach the target location.

15. The method of claim 14, further comprising deploying a medical implant to the target location using the guidewire.

16. The method of claim 13, further comprising after deploying the guidewire, withdrawing the hollow needle from the target location while retaining the hollow needle in the catheter.

17. The method of claim 13, wherein the hollow needle is made at least partly from nylon or nitinol.

18. A hollow needle for delivering a guidewire to a target location in a patient's body, the hollow needle comprising:
a lumen configured to receive the guidewire;
a body section of the hollow needle; and
a biased section of the hollow needle, the biased section configured to bend relative to the body section, the biased section located on a distal end of the hollow needle.

19. The hollow needle of claim 18, further comprising:
a second lumen configured to hold a wire stiffener, the wire stiffener comprising a pointed end configured to pierce tissue;
wherein the body section and the biased section are made from a plastic material and the wire stiffener is configured to maintain the relative bend of the biased section to the body section.

20. The hollow needle of claim 18, further comprising a plurality of cuts made in the biased section, the plurality of cuts configured to allow the biased section to bend.
